(19) United States
(12) Patent Application Publication
(10) Pub. No.: US 2024/0324935 A1
(43) Pub. Date: Oct. 3, 2024

(54) Portable Lead Smartwatch Electrocardiography Apparatus and Methods of Use

(71) Applicant: Luiz Maracaja, Winston Salem, NC (US)

(72) Inventor: Luiz Maracaja, Winston Salem, NC (US)

(21) Appl. No.: 18/623,004

(22) Filed: Mar. 31, 2024

Related U.S. Application Data
(60) Provisional application No. 63/456,188, filed on Mar. 31, 2023.

(51) Int. Cl. A61B 5/332; A61B 5/00; A61B 5/256; A61B 5/26; A61B 5/282; A61N 1/39

(52) U.S. Cl. CPC A61B 5/332; A61B 5/0006; A61B 5/0022; A61B 5/256; A61B 5/26; A61B 5/282; A61B 5/681; A61B 2560/0214; A61B 2560/0443; A61B 2562/222; A61B 2562/227; A61N 1/3904

(57) ABSTRACT

Certain embodiments are directed to an electrocardiogram system or components of a system, the system comprising a smartwatch configured to perform an electrocardiogram of a first subject wearing the smartwatch; an electrocardiogram adapter, the adapter configured to interface smartwatch sensors with an electrocardiogram lead; and an electrocardiogram lead operatively coupled to the smartwatch by the electrocardiogram adapter, wherein the system is configured to perform an electrocardiogram of a second subject that is not wearing the smartwatch.

### FIELD OF INVENTION

The present invention relates to the field of medicine, in particular the use of wearable devices to monitor health functions in particular devices that can provide electrocardiogram.

### BACKGROUND

ECG stands for Electrocardiogram, which is a medical test that records the electrical activity of the heart. Electrocardiography is a process to produce an electrocardiogram, also known as ECG or EKG. Electrocardiogram is a recording of the heart's electrical activity through repeated cardiac cycles. The electrocardiogram is displayed as a graph of voltage versus time of the electrical activity of the heart using pairs of electrodes placed on the surface of the skin, or internally. The 12 lead ECG test involves attaching electrodes to the skin of the chest, arms, and legs.

The electrodes detect small electrical changes that result from the depolarization and repolarization of the heart during each cycle. In a conventional ECG or 12-lead ECG, ten electrodes are placed on the patient's limbs and the surface of the chest. The heart's electrical activity is measured from twelve different locations or leads and is recorded over time. ECG electrodes and cables are used to measure the electrical activity of the heart. The electrodes are most commonly placed on the skin and are connected to the ECG machine through cables. The ECG machine then records the electrical activity of the heart, which is represented graphically as an ECG waveform.

The electrodes are typically small, sticky patches that are attached to the skin using a conductive gel. The cables connect the electrodes to the ECG machine and transmit the electrical signals from the electrodes to the machine. There are several types of ECG electrodes and cables, including disposable and reusable options. Disposable electrodes are typically used in hospital settings and are designed to be used once and then discarded. Reusable electrodes are designed to be used multiple times and are often used in outpatient settings. The cables that connect the electrodes to the ECG machine can vary in length, and some ECG machines may require specific types of cables. It is important to use the correct cables and electrodes to ensure accurate ECG readings.

During each cardiac cycle or heartbeat, there is a sequence of events according with the progression of depolarization that stars with the sino-atrial node, progresses through the right and left atrium, crosses, and slows conduction through atrioventricular node, goes into the bundle of Hiss and Purkinge fibers that spread through the myocardium of the left and right ventricle. This sequential pattern generates the characteristic ECG tracing, with three main components that represents, depolarization of the atria (P wave), depolarization of the ventricles (QRS complex); and the repolarization of the ventricles (T-wave). This graph over time conveys a large amount of information about the function of the heart, the effects of drugs, or function of pacemakers. Changes in the normal ECG pattern occur in numerous cardiac abnormalities, including cardiac rhythm disturbances or arrhythmias, inadequate coronary blood flow or coronary artery disease, severe hypothermia, and electrolyte disturbances.

Einthoven's triangle is a theoretical concept in the field of electrocardiography. It is a triangle that is formed by the three limb leads used in a standard ECG: lead I, lead II, and lead III. These leads are placed on the patient's limbs, with lead I on the right arm, lead II on the left leg, and lead III on the left arm. The Einthoven's triangle is named after Willem Einthoven, a Dutch physiologist who developed the ECG machine and contributed significantly to the field of electrocardiography. The Einthoven's triangle is important because it allows for the calculation of the electrical activity of the heart in three dimensions, even though the ECG leads are placed on the body's surface. The triangle's sides are equal to the electrical potentials generated by the heart, and the angles between the sides provide information about the direction of the electrical current flow within the heart. This information can be used to diagnose problems in specific parts of the heart.

The electrodes are connected by cables to an ECG machine to record, the early ECG machines were constructed with analog electronics where the electric signal would drive a motor to print out the signal onto paper. The newer machines use converters from analog to digital, converting the cardiac electric activity into a digital signal. ECG machines are now portable and commonly include a screen, keyboard, and printer.

The conversion of digital signal allowed the use of computer algorithms to perform automated analysis of electrocardiogram, storage in computer data and wireless transmission. Several advancements in electrocardiography, resulted in smaller devices and now the electrocardiogram is available in wearable devices such as smartwatches.

A smartwatch is a wearable mini-computer in the form of a watch. Smartwatches are normally associated wirelessly with smartphones via Bluetooth or WIFI. The smartwatch has a touchscreen interface and allows navigation of multiple applications for daily tasks, Some Smartwatches carry cellular signal allowing for making phone calls, geolocation tracking and wireless transmission of data.

Wearable health devices offer several benefits to users and healthcare providers. Smartwatches have become increasingly popular in recent years as a convenient way to track one's health and fitness. One of the features that many modern smartwatches offer is the ability to record and analyze electrocardiogram (ECG) readings. This allows individuals to track their heart health in real-time and potentially detect any irregularities that may require medical attention.

Smartwatches offer a convenient way to measure, monitor, store and share health information. They provide feedback to users to change behavior and allow real time remote patient monitoring. Smartwatches can potentially detect life-threatening arrhythmias and provide real-time or faster data access to physicians. Different technology present in smartwatches allow the measurements of ECG, and calorie consumption during exercise, and attempts have been made to monitor glucose levels without needle sticks for blood samples. Smartwatches are particularly useful for patients with diabetes, cardiopulmonary disease, and other chronic health conditions. The watch then records the heart's electrical activity for a specified period, usually between 30 seconds to a minute. The recorded data can then be analyzed by the watch or uploaded to a smartphone app for further analysis.

Currently to use the ECG feature on a smartwatch, the user typically needs skin contact with two electrodes, one on each arm of the user. Usually, for the watch worn on the left arm, the left arm electrode is located on the undersurface of the watch, and the right arm electrode is on a designated area on the side of the watch, requiring the user to place a right-hand finger on the designated area.

The current smartwatch setup was designed to obtain ECG only from the person using the watch. If a physician or health care provider using the watch desires to obtain the ECG from another person or a patient, the first will have to remove the watch and apply it to the arm of the patient. Exchanging a personal watch is not a commonly acceptable habit for various reasons. Moreover, in a hospital environment, removing the watch from a physician and applying the watch to multiple patients would result in the transmission of skin germs and the need to constant washing and disinfect the watch.

The portable ECG technology would be beneficial, if the smartwatch could also perform an ECG from another subject not using the watch. This could facilitate physicians and heath care providers to obtain immediate heart hate and rhythm analysis of patients as part of the physical exam. Moreover, the technology could be life-saving in situations where patients are unconscious and unable to obtain a formal EC, such as in remote locations or austere environments. Sudden cardiac death commonly affects hundreds of thousands of people yearly. Initiation of cardiopulmonary resuscitation (CPR) and early defibrillation are critical for survival, yet many bystanders are reluctant to intervene. Digital technologies might help play a role in improving survival from lethal arrhythmia. Cardiac arrest is a life-threatening medical emergency in which the heart suddenly stops beating. This can occur because of a variety of factors, including arrhythmias. Early cardioversion, which involves restoring the heart's normal rhythm using electrical shocks, can be an effective treatment for cardiac arrest caused by certain arrhythmias. Malignant arrhythmias are treated with defibrillation. Defibrillation is a strong shock that crosses the heart, and it provides resynchronization of the myocardium rhythm and return of spontaneous circulation.

Arrhythmias are a common medical condition in which the heart beats irregularly, or too fast. This can lead to several health problems, including chest pain, shortness of breath, and fainting. One of the most effective treatments for arrhythmias is early cardioversion, which involves restoring the heart's normal rhythm as soon as possible after the onset of symptoms. The goal of early cardioversion in cardiac arrest is to restore the heart's normal rhythm as quickly as possible to increase the chances of survival. When the heart stops beating, blood flow to the brain and other organs is interrupted, which can cause irreversible damage within minutes. Early cardioversion can help restore blood flow and prevent further damage to vital organs. In order to determine if early cardioversion is appropriate for cardiac arrest caused by an arrhythmia, healthcare providers will typically perform an electrocardiogram (ECG) to determine the type and severity of the arrhythmia. If the arrhythmia is determined to be treatable with cardioversion, electrical shocks will be delivered to the heart to restore its normal rhythm. Early cardioversion in cardiac arrest is most effective when performed within the first few minutes after the onset of symptoms. For this reason, it is critical for healthcare providers to act quickly in response to a cardiac arrest event. Prompt treatment with early cardioversion can significantly improve the chances of survival and reduce the risk of long-term complications. In summary, early cardioversion can be an effective treatment for cardiac arrest caused by certain types of arrhythmias. It is critical for healthcare providers to act quickly and perform early cardioversion as soon as possible to increase the chances of survival and reduce the risk of long-term complications. The goal of early cardioversion is to restore the heart's normal rhythm before the arrhythmia can cause further problem to the heart and brain. Over time, arrhythmias can become more frequent and more severe, leading to complications such as heart failure, stroke, and sudden cardiac death. By restoring the heart's normal rhythm early on, cardioversion can help prevent these complications and improve overall outcomes. Another benefit of early cardioversion is that it can help alleviate symptoms associated with arrhythmias, such as chest pain, shortness of breath, and dizziness. These symptoms can be debilitating and can greatly impact a patient's quality of life. By restoring the heart's normal rhythm, early cardioversion can help alleviate these symptoms and improve overall well-being. Early cardioversion is also associated with a lower risk of complications compared to delayed cardioversion. When the heart beats irregularly for an extended period, it can cause damage to the heart muscle and increase the risk of blood clots, stroke, and other complications. Cardioversion can help reduce these risks and improve overall outcomes by restoring the heart's normal rhythm early on. In summary, cardioversion is a lifesaving treatment for many types of arrhythmias, and during cardiac arrest, every second counts to determine the arrhythmia and have access to a defibrillator.

Manual defibrillators are a special type of equipment present only in heath care facilities and medical transport and managed by health care providers. Automatic external defibrillators, or AEDs, are devices that can be used by non-healthcare providers and require minimal training. Such devices can perform ECG and determine if there is a need for cardioversion. There are usually located in areas of high circulation, such as, shopping malls, offices, schools, grocery stores, and airports. Unfortunately, many victims of sudden death and arrhythmias will not survive because they don't have timely access to a defibrillator.

### SUMMARY OF THE INVENTION

The present invention seeks to provide a solution to these problems by providing a smartwatch, smartphone, or watch cover with frontal electrodes to the health care provider can perform the ECG of patients. The additional connector for a cable system provides additional secondary electrodes to be positioned on the surface of the patient skin.

The embodiments here described can be part of the smartwatch or watch cover that is positioned on the outside of the watch. The position of two separate electrodes in the front face would allow the user to visualize the ECG of a patient or subject by looking at the screen on the top face of the watch while the patient or subject places the left hand finger and right-hand finger in the designated areas of the watch.

Certain embodiments are directed to an electrocardiogram system comprising: (a) a smartwatch configured to perform an electrocardiogram of a first subject wearing the smartwatch; (b) an electrocardiogram adapter, the adapter configured to interface smartwatch sensors with an electrocardiogram lead; and (c) an electrocardiogram lead operatively coupled to the smartwatch by the electrocardiogram adapter; wherein the system is configured to perform an electrocardiogram of a second subject that is not wearing the smartwatch. The electrocardiogram lead can be configured with distal electrodes to be placed on the skin surface of the second subject. The system or the electrocardiogram lead can further comprise defibrillator pads coupled to the electrocardiogram lead. The system can be configured for direct transmission by wire or wireless transmission of electrocardiogram data to a remote device such as a tablet or other monitoring system or remote device (remote device being a device not part of the system for set of components used to obtain the electrocardiogram data from the subject. The remote device can be in a physician's office or an emergency services terminal or device. The electrocardiogram lead can be configured as a reel container having an electrocardiogram lead having a proximal magnetic connector and one or more distal electrodes with a cable connecting the magnetic connector, wherein the cable is retractably wound on a reel of the reel container. The system can be configured to register one or more electrocardiogram leads simultaneously or sequentially. The sequential assessment can use less than 12 leads to query one or more lead placements sequentially with the data being manipulated to generate an electrocardiogram. The system can include a software component for guiding placement of electrodes in specific positions and includes the capability of registration, and graphic construction of a 12 lead ECG.

Certain embodiments are directed to an electrocardiogram adapter for a smartwatch or a smartphone that is configured to connect sensors on the smartwatch or smartphone to electrocardiogram electrodes, connections to the electrocardiogram adapter being positioned on the sides or top of the smartwatch or smartphone to provide an electrocardiogram lead connection when the smartwatch is being worn by a user or the smartphone is placed backside down on a surface or a front side is being viewed by a user. The adapter can be configured as a cover that removably attaches to the smartwatch or the smartphone. The adapter having a conductive path that connects a devices sensors to an electrocardiogram lead attachment point that is accessible when the smartphone is being worn by a user to monitor a third person.

Certain embodiments are directed to an electrocardiogram lead coupled to a reel container: the electrocardiogram lead having a proximal magnetic connector, one or more distal electrodes, and a cable connecting the magnetic connector and the electrodes, wherein the cable is retractably and reversibly wound on the reel. The reel container can be a wearable container configured to clip or attached to the clothing of a user.

Certain embodiments are directed to a portable defibrillator configured to operatively connect with a power bank or battery, the power bank or battery being configured to charge handheld devices. The portable defibrillator can be configured to connect to a smartwatch, or a smartphone.

The location of the electrodes and connector in the front face, front top face, or edges allows the connection with a cable system that can be extended the cable and placement of electrodes on patient's skin surface, allowing different lead configurations different than D2. The ability to register ECG from different electrode positions allows the acquisition of peripheral and precordial leads. If electrodes are placed sequentially to specific locations correspondent to classic 12 lead ECG, the system can sequentially record all the 12 leads. In certain aspects a software application on the smartphone provides procedure instructions in audible, written, and graphic form, audible and written form; audible and graphic form; or written and graphic form using the smartphone's speakers, graphical user interface, or other capabilities of the smartphone.

The location of the electrodes and connector in the front face or front top face allows the connection with a cable system that allows the position in the patient's skin surface, allowing different lead configurations different than D2.

This feature also allows the acquisition of ECG in patients that are unconscious and unable to position fingers in specific locations or obtain all the lead configurations from a traditional ECG.

The preferred embodiment of the described invention, the cable system has two permanent electrodes that are functional with or without electrolytic gel or adhesive (sticker) in one extremity. The cable is built with a coaxial configuration to avoid background electric noise from motion. The other extremity is built with a connector with magnetic alignment of the electrodes. The cable is contained in a dual retractable cable system that can be used as or combined with the retractable system or reel system from ID badges. This feature would allow all health professionals to have the cable available if ECG is required.

The placement of electrodes on the front surface or cable connected allow versatility and to perform the ECG by patient placing fingers on the front surface, or by connecting cables and placing electrodes on the patients' skin surface.

These features allow acquisition of the ECG and analysis in a very short period of time they would benefit patients with the identification of cardiac arrhythmias and establish treatment.

Once the ECG recognizes s malignant or life-threatening arrhythmia, allowing medical professionals to make decisions related to the treatment. The built-in algorithm perform recognition and gives verbal directives to guide bystanders at the scene of cardiac arrest if a medical professional is not present.

An additional embodiment of the invention is a portable pocket size defibrillator and defibrillating pads that are connected to connected to the smartwatch and smartphone, to provide acquisition of the cardiac rhythm, ECG analysis and manual or automatic defibrillation. The portable defibrillator would have an additional set of rechargeable batteries that could be used as a power bank to charge smartphones and other portable devices. The portable defibrillator would be available more often available to rescue victims of cardiac arrest and could be easily recharged in regular power walls or automobiles or other vehicles.

### BASIC DESCRIPTION OF THE DRAWINGS

FIG. 1. ECG tracing for one cardiac cycle and respective events

FIG. 2. Traditional 12 lead electrocardiogram

FIG. 3. The position chart for leads for traditional ECG, or 12 lead ECG.

FIG. 4. ECG machine and cables connected to the chest wall.

FIG. 5. Triangle of Einthoven

FIG. 6 Top view of Smartwatch

FIG. 7. Bottom view of the smartwatch.

FIG. 8. ECG acquisition using a currently available smartwatch, ECG from the user of the smartwatch.

FIG. 9. Top view of the smartwatch with frontal electrodes and connector for ECG cable.

FIG. 10. Perspective view of the smartwatch with frontal electrodes.

FIG. 11. Top view of smartwatch on the hand of the user.

FIG. 12. Top view of smartwatch on the hand of the user performing ECG from a patient.

FIG. 13. Top view of Smartwatch cover with frontal electrodes and cable connector.

FIG. 14. Perspective view of Smartwatch cover with frontal electrodes and cable connector.

FIG. 15. Perspective view of smartwatch with cover with frontal electrodes and connector.

FIG. 16. Side view of smartwatch with cover with frontal electrodes and connector for ECG lead,

FIG. 17 Perspective view of ECG lead cable within the retractable reel.

FIG. 18 Perspective view ECG cable and retractable reel attached with ID badge.

FIG. 19 ECG lead connection between the patient and smartwatch

FIG. 20. Defibrillating and ECG paddles, portable defibrillator, and Smartwatch.

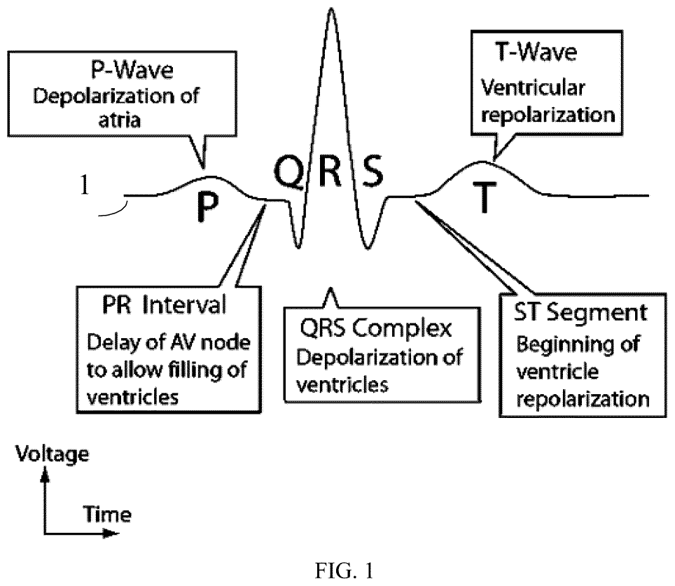

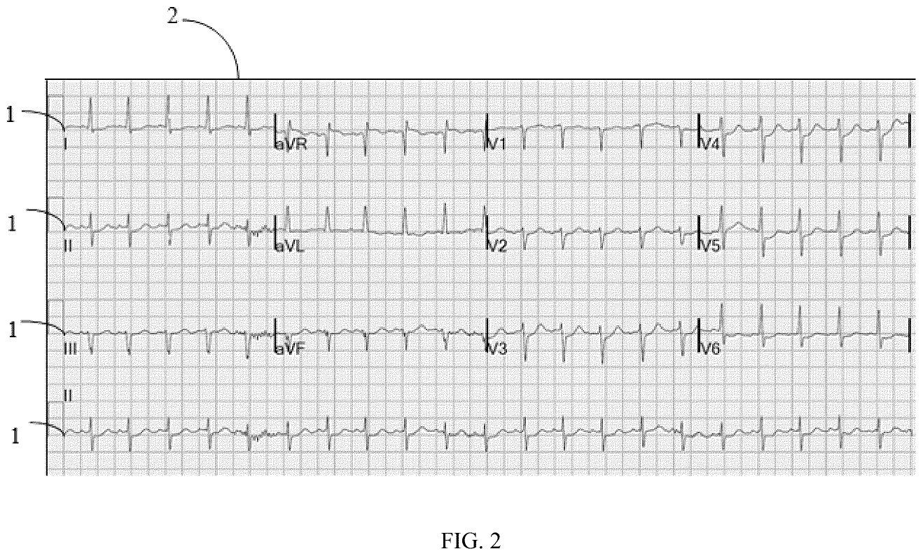

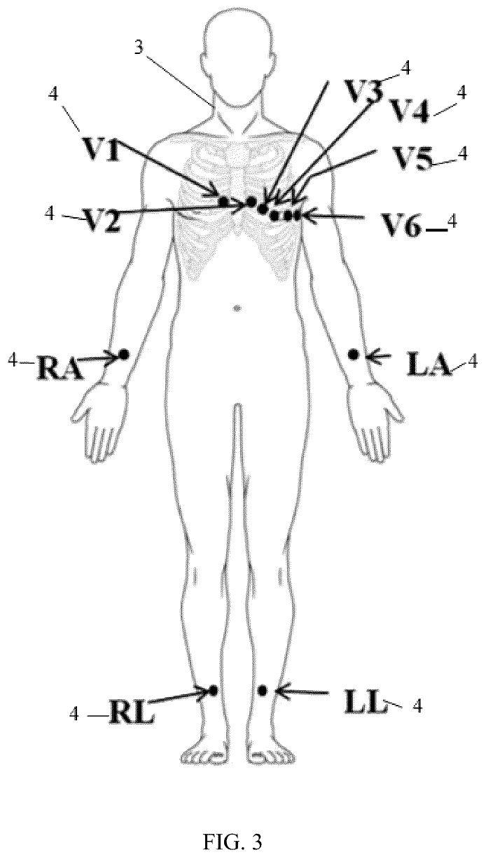

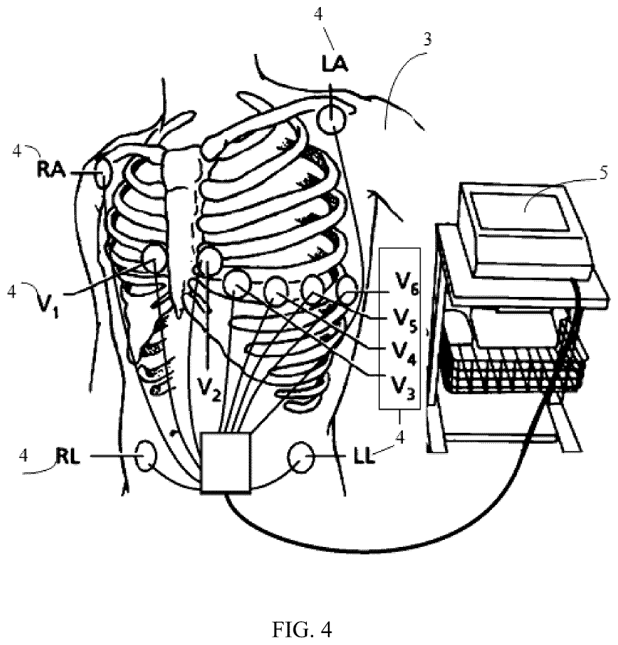

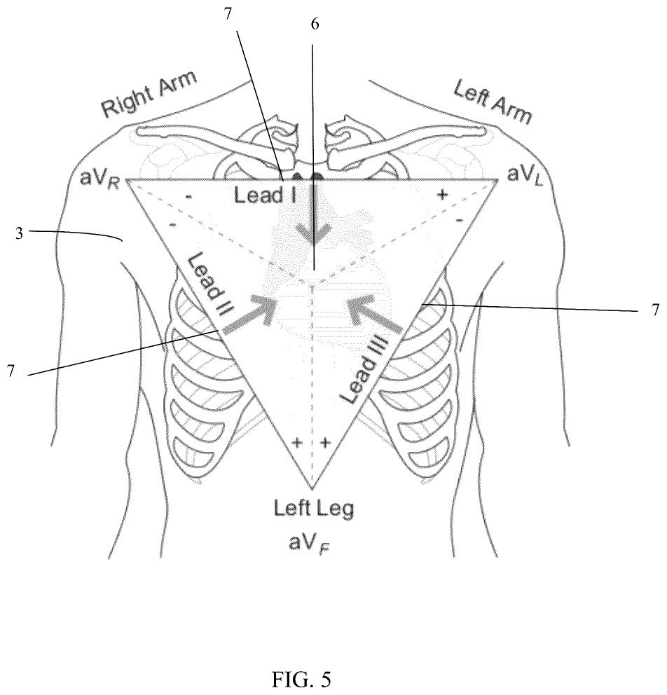

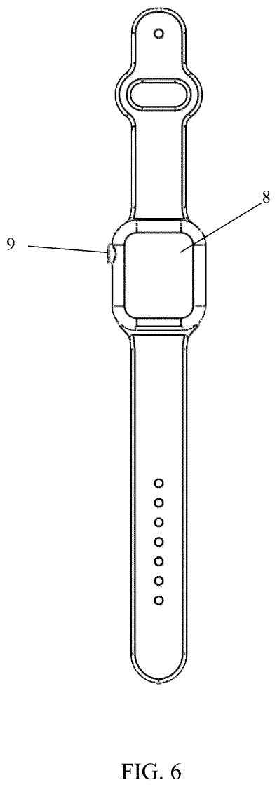

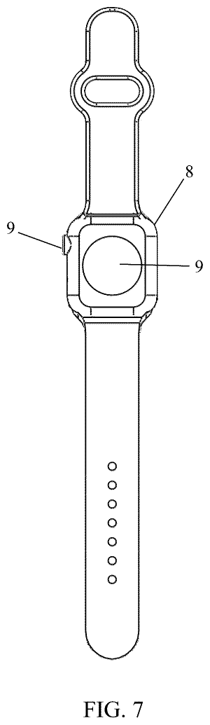

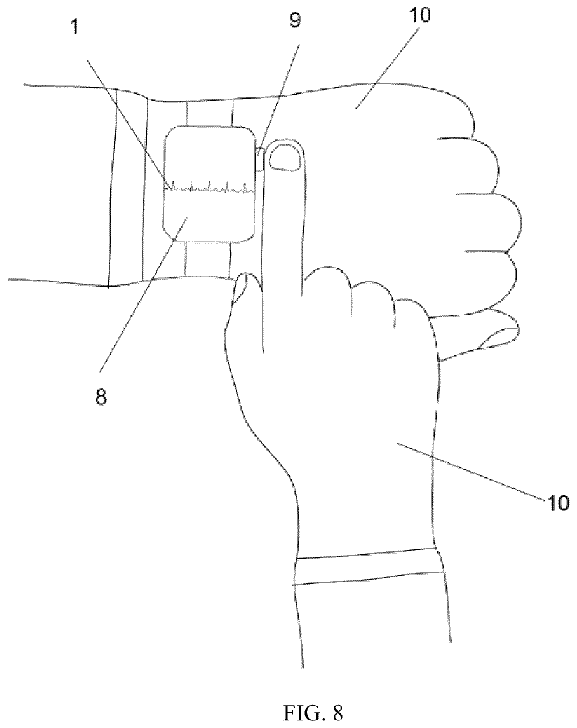

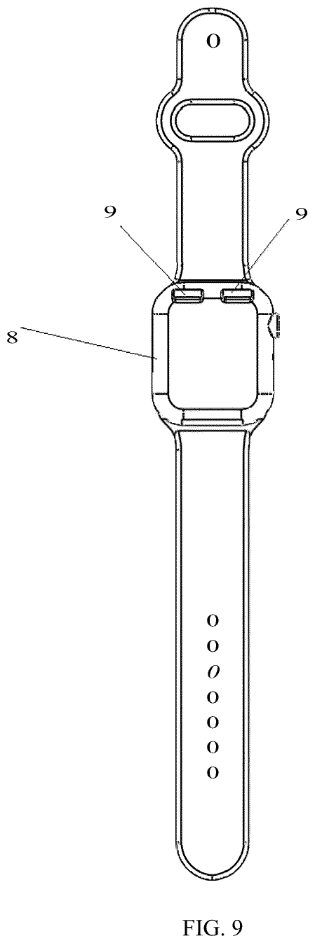

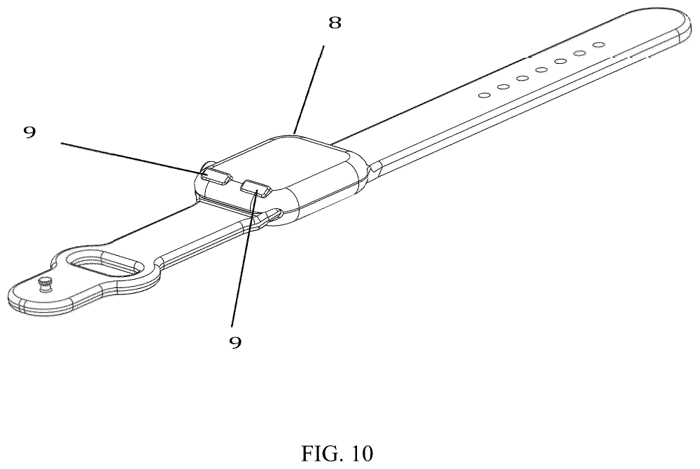

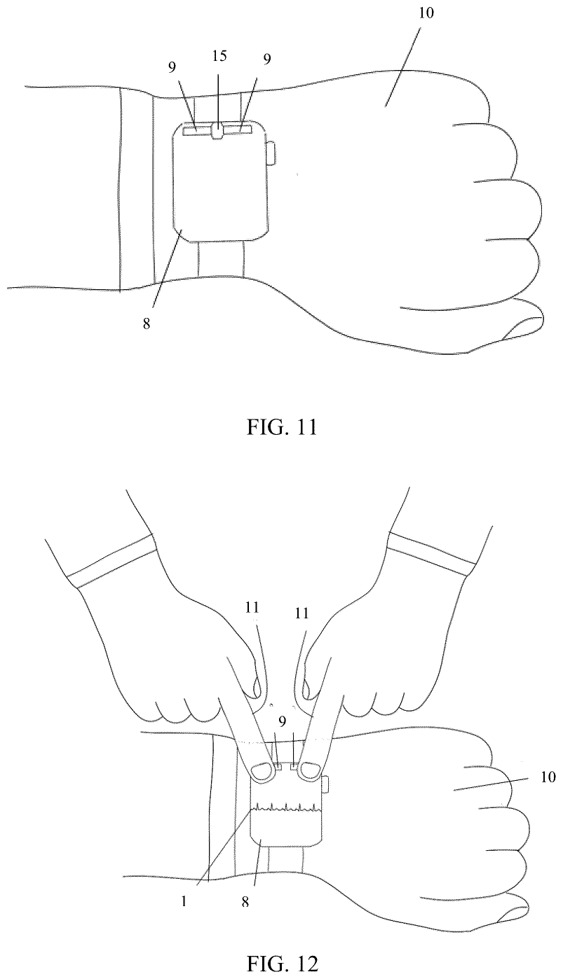

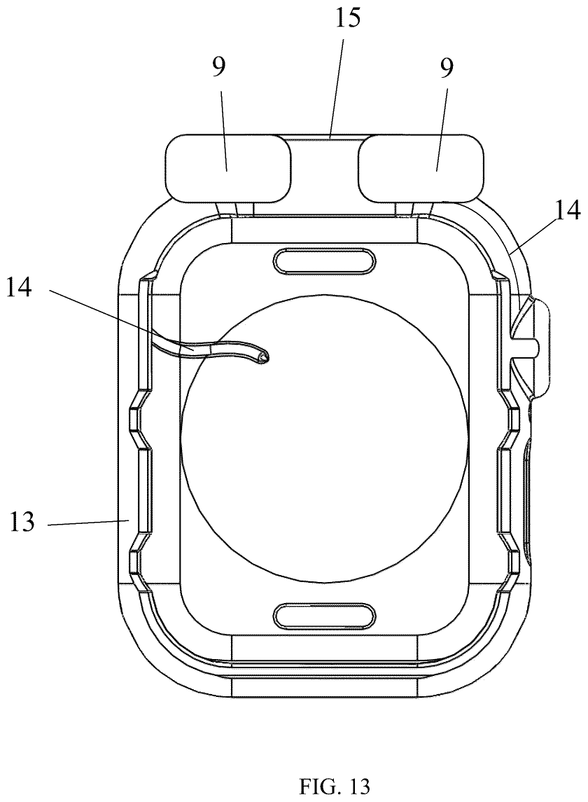

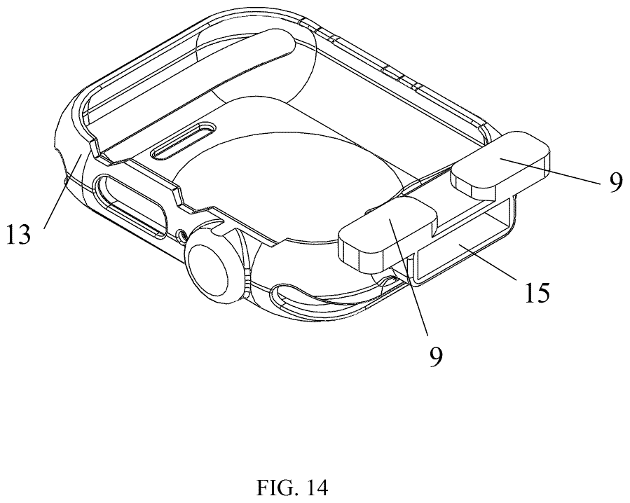

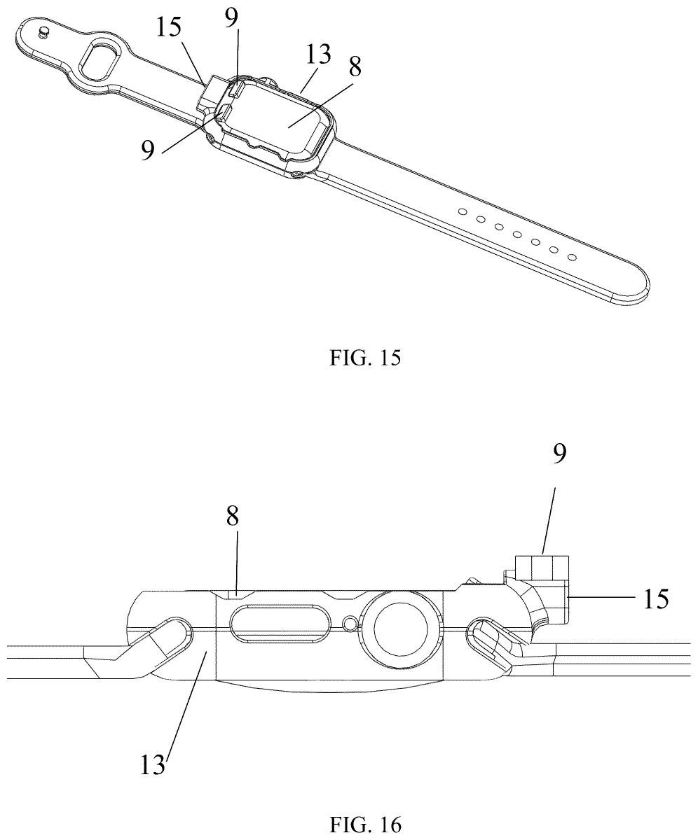

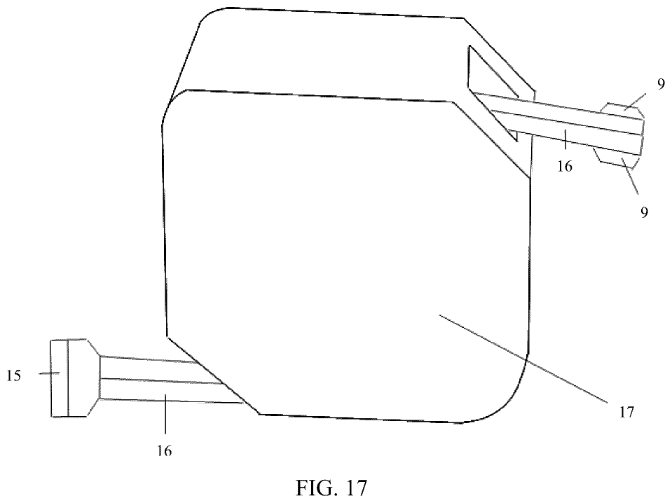

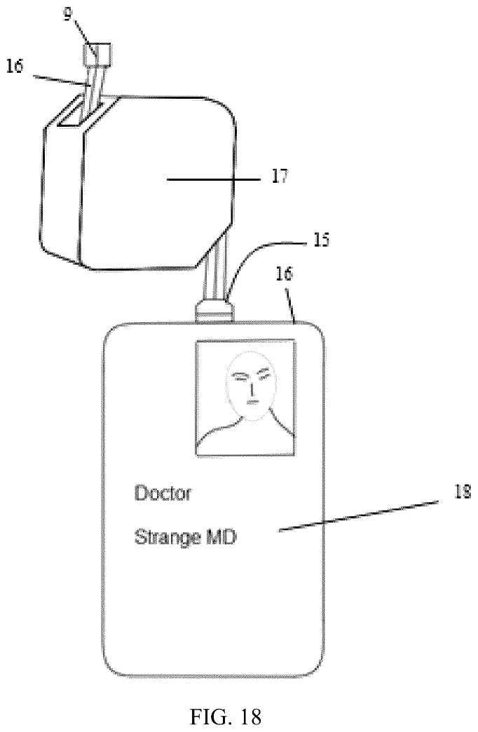

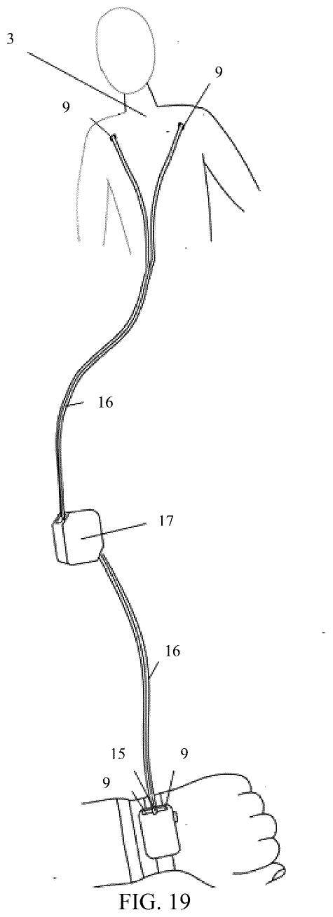

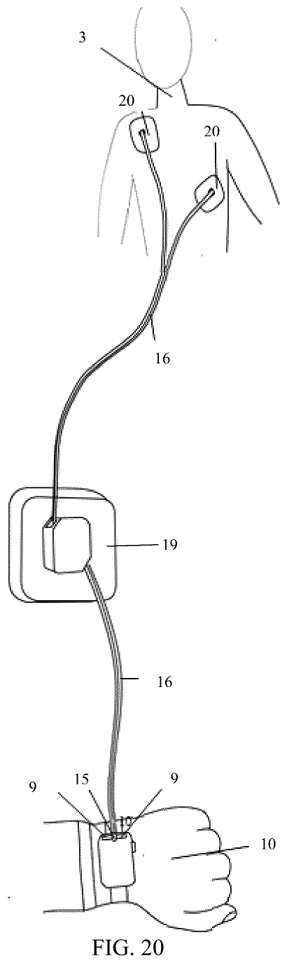

1—ECG tracing
2-12 lead electrocardiogram.
3—Patients body representation
4—Leads
5—ECG machine
6—Einthoven triangle with respective leads
7—Representation of leads position and direction
8—smartwatch
9—Electrodes
10—the user's hands
11—the patient's fingers
13—Cover for watch
14—wires
15—Connector for Cable
16—Cable
17—Reel for cable connector
18-1D Badge
19—Defibrillator
20—Defibrillating Pads.

1. An electrocardiogram system comprising:
(a) a smartwatch configured to perform an electrocardiogram of a first subject wearing the smartwatch;
(b) an electrocardiogram adapter, the adapter configured to interface smartwatch sensors with an electrocardiogram lead; and
(c) an electrocardiogram lead operatively coupled to the smartwatch by the electrocardiogram adapter;
wherein the system is configured to perform an electrocardiogram of a second subject that is not wearing the smartwatch.

2. The system of claim 1, wherein the electrocardiogram lead is configured with distal electrodes to be placed on the skin surface of the second subject.

3. The system of claim 1, further comprising defibrillator pads coupled to the electrocardiogram lead.

4. The system of claim 1, configured for wireless transmission of data to a remote device.

5. The system of claim 4, wherein the remote device is in a physician's office or an emergency services terminal or device.

6. The system of claim 1, wherein the electrocardiogram lead is configured as a reel container having a proximal magnetic connector and one or more distal electrodes with a cable connecting the magnetic connector, wherein the cable is retractably wound on a reel of the reel container.

7. The system of claim 1, wherein the system is configured to register one or more electrocardiogram leads.

8. The system of claim 7, comprising a software component for guiding placement of electrodes in specific positions, registration, and graphic construction of a 12 lead ECG.

9. An electrocardiogram adapter for a smartwatch or a smartphone that is configured to connect sensors on the smartwatch or smartphone to electrocardiogram electrodes, connections to the electrocardiogram adapter being positioned on the sides or top of the smartwatch or smartphone to provide an electrocardiogram lead connection when the smartwatch is being worn by a user or the smartphone is placed backside down on a surface or a front side is being viewed by a user.

10. The adapter of claim 9, wherein the adapter is configured as a cover that removably attaches to the smartwatch or the smartphone.

11. An electrocardiogram lead comprising a reel container, a magnetic connector, one or more electrodes, and a cable connecting the magnetic connector, wherein the cable is retractably wound on the reel.

12. The electrocardiogram lead of claim 11, wherein the reel container is a wearable container configured to clip or attached to the clothing of a user.

13. The system of claim 1, further comprising a portable defibrillator configured to operatively connect with a power bank or battery, the power bank or battery being configured to charge handheld devices.

14. The portable defibrillator of claim 13, configured to connect to a smartwatch, or a smartphone.
